(19) United States
(12) Patent Application Publication
(10) Pub. No.: US 2025/0250385 A1
(43) Pub. Date: Aug. 7, 2025

(54) PRODUCTION OF POLYURETHANE OR POLYISOCYANURATE FOAM

(71) Applicant: Evonik Operations GmbH, Essen (DE)

(72) Inventor: Martin Glos, Borken (DE)

(73) Assignee: Evonik Operations GmbH, Essen (DE)

(21) Appl. No.: 19/041,358

(22) Filed: Jan. 30, 2025

(30) Foreign Application Priority Data
Feb. 2, 2024 (EP) 24155463.3

(51) Int. Cl. C08G 18/08; C07C 209/60; C08G 18/09; C08G 18/22; C08G 18/42; C08J 9/00; C08J 9/12; C08J 9/14; C08K 5/09; C08K 5/17

(52) U.S. Cl. CPC C08G 18/0838; C07C 209/60; C08G 18/092; C08G 18/222; C08G 18/42; C08J 9/0028; C08J 9/125; C08J 9/141; C08K 5/09; C08K 5/17; C08G 2110/0025; C08J 2205/10; C08J 2375/06

(57) ABSTRACT

A composition for producing polyurethane or polyisocyanurate foam has a polyisocyanate component, a polyol component, at least one tertiary amine of formula (X) and at least one zinc(II) carboxylate with the proviso that the zinc(II) carboxylate present is employed in stoichiometric form, i.e. with Zn(II) and carboxylate in a molar ratio of 1 to 2, wherein the composition additionally contains at least one nitrogen-containing compound V which comprises at least two N atoms and which is a modified phenol.

### CROSS-REFERENCE TO RELATED APPLICATIONS

This patent application claims priority to European Patent Application No. 24155463.3, filed on Feb. 2, 2024, in the European Patent Office, the entire disclosure of which is hereby incorporated by reference herein.

### BACKGROUND OF THE INVENTION

### Field of the Invention

The present invention is in the field of polyurethanes (PU) and polyisocyanurates (PIR), especially of PU or PIR foams. It especially relates to a composition for producing polyurethane or polyisocyanurate foam, to a zinc(II) carboxylate-containing preparation suitable for catalysis in the production of PU or PIR foam, to a process for producing polyurethane or polyisocyanurate foam and to polyurethane or polyisocyanurate foam, and the use of the foams, produced therewith.

Polyurethane (PU) in the context of the present invention is especially understood to mean a product obtainable by reaction of polyisocyanates and polyols or compounds having isocyanate-reactive groups. In addition to the polyurethane, further functional groups may also be formed here, for example uretdiones, carbodiimides, isocyanurates, allophanates, biurets, ureas and/or uretonimines. For the purposes of the present invention, PU is therefore understood to mean not just polyurethane, but also polyisocyanurate, polyureas, and polyisocyanate reaction products containing uretdione, carbodiimide, allophanate, biuret and uretonimine groups. In the context of the present invention polyurethane foam (PU foam) is especially understood to mean foam obtained as a reaction product based on polyisocyanates and polyols or compounds having isocyanate-reactive groups. In addition to the eponymous polyurethane, further functional groups can be formed as well, examples being allophanates, biurets, ureas, carbodiimides, uretdiones, isocyanurates or uretonimines. In the context of the present invention polyisocyanurate foam (PIR foam) is especially understood to mean a foam which is obtained as a reaction product based on polyisocyanates and polyols or compounds having isocyanate-reactive groups and where the isocyanate index, i.e. the ratio between the isocyanate and isocyanate-reactive groups, is preferably greater than 180. In addition to the eponymous polyisocyanurates, polyurethane groups and possibly further functional groups may well be formed here as well, examples being allophanates, biurets, ureas, carbodiimides, uretdiones, or uretonimines.

In the context of the present invention it is preferably the formation of polyisocyanurates (PIR) that is the focus. This reaction is referred to as trimerization since, in a formal sense, three isocyanate groups react to give an isocyanurate ring. The production of for example rigid PIR foam is described in the literature and is preferably effected by reacting polyisocyanates with compounds having hydrogen atoms reactive toward isocyanate groups, typically polyetherols, polyesterols or both, where the isocyanate index is preferably 180 or greater. In addition to the urethane structures formed by the reaction of isocyanates with compounds having reactive hydrogen atoms, this results in formation, via reaction of the isocyanate groups with one another, of isocyanurate structures or further structures that result from the reaction of isocyanate groups with other groups, for example polyurethane groups.

The production of polyurethane or polyisocyanurate foams, preferably rigid polyurethane or polyisocyanurate foams, may employ different catalysts to positively affect the reaction profile of the foaming and the performance characteristics of the foam. The formation of polyisocyanurates is advantageous here since these lead to good mechanical properties (high compressive strength) and improved flame retardant properties.

### Description of Related Art

There are various known publications relating to the use of catalysts for improvement of compressive strength by promoting the trimerization reaction in the production of rigid PU or PIR foams.

EP 1878493 A1 describes the use of carbocationic compounds as trimerization catalysts, wherein the anions are based on dicarbonyl compounds. The use of zinc carboxylates is not described.

U.S. Pat. No. 4,452,829 describes the production of spray foam using triols having molar masses exceeding 1000 g/mol. This comprises employing Zn salts in conjunction with K salts to accelerate creaming, that is to say commencement of the PU reaction with water, i.e a K-containing catalyst is admixed with a Zn-containing catalyst (zinc octoate) to shorten the cream time, i.e. to accelerate the reaction.

U.S. Pat. No. 4,200,699 describes gel catalyst compositions for the production of rigid PU foams containing zinc carboxylates, potassium carboxylates and tin carboxylates, preferably with use of a further gel catalyst from the group of the tertiary amines, the inorganic tin compounds or the organotin compounds.

EP 1 745 847 A1 describes trimerization catalysts based on potassium oxalate and solvents that are inert with respect to the reaction with isocyanates.

WO 2016/201675 A1 describes trimerization catalysts consisting of compositions based on sterically hindered carboxylates and tertiary amines that bear an isocyanate-reactive group.

WO 2010/054317 A2 describes imidazolium or imidazolinium salts as trimerization catalysts.

WO 2013/074907 A1 describes the use of tetraalkylguanidine salts of aromatic carboxylic acids as catalysts for polyurethane foams.

WO 2015/179041 A1 describes the use of zinc-based catalysts for crosslinking of silicone resins. Different ligands are employed here, inter alia phenol-based ligands. However catalysis of a polyurethane or isocyanate reaction is not described.

WO 2022/218657 A1 describes the production of rigid polyurethane or polyisocyanurate foam using zinc salts and/or a zinc-containing preparation.

US 2009/099274 A1 discloses rigid PU or PIR foam produced from a composition comprising an isocyanate component, a polyol component, a blowing agent, amine-based catalyst and zinc catalyst.

WO 2019/122923 A1 discloses rigid PU or PIR foam produced from polyisocyanate and polyether carbonate and zinc-based catalyst.

EP 0 010 407 A1 discloses a process for producing PU foam in the presence of gel catalysts containing zinc carboxylates.

### SUMMARY OF THE INVENTION

The problem addressed by the present invention was that of enabling a further means of providing polyurethane or polyisocyanurate foams, preferably rigid polyurethane or polyisocyanurate foams, which have particularly advantageous performance characteristics, such as especially good compressive strength and/or indentation hardness after only a short reaction time. However, the effect on the rise profile is preferably to be kept as low as possible.

### DETAILED DESCRIPTION OF THE INVENTION

The solution to this problem is brought about by the subject matter of the invention.

The present invention provides a composition for producing polyurethane or polyisocyanurate foam, preferably rigid polyurethane or polyisocyanurate foam, comprising
a) a polyisocyanate component,
b) a polyol component,
c) at least one zinc(II) carboxylate with the proviso that the zinc(II) carboxylate present is employed in stoichiometric form, i.e. with Zn(II) and carboxylate in a molar ratio of 1 to 2,
d) at least one tertiary amine of formula (X)

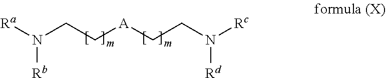

where
m is independently at each occurrence 1 or 2,
A is O, S or N—R^{e},
R^{a}, R^{b}, R^{c}, R^{d} and R^{e} are each independently of one another identical or different linear, branched or cyclic alkyl radicals having 1 to 20 carbon atoms,
e) optionally at least one foam stabilizer,
f) optionally at least one blowing agent, wherein the composition additionally contains
g) at least one nitrogen-containing compound V which comprises at least two N atoms and which is a modified phenol.

It has been found that the use of compositions according to the invention in PU or PIR foam production, preferably rigid PU or PIR foam production, results in corresponding foams, preferably rigid foams, having improved performance characteristics. Trimerization is especially improved with the result that the foams cure quickly, i.e. already have a high compressive strength and high indentation hardness at an early juncture.

It is also a particular advantage of the present invention that the use of the compositions according to the invention nevertheless enables minimization of the influence on the rise profile. This is very advantageous since problems can otherwise occur with the flowability of the reaction mixture, which leads to considerable processing problems.

With the compositions according to the invention, it is in some cases also possible to slow the rise profiles, which enables a wide variety of options for adjusting the reactivity of a foam system.

The solution according to the invention thus makes it possible to produce products based on PU or PIR foam, preferably rigid PU or PIR foam, for example insulation panels or refrigeration equipment in particularly high quality and to make the processes for producing the PU or PIR foams, preferably rigid PU or PIR foams, more efficient.

An additional advantage of the invention is the good environmental toxicology classification of the employable chemicals, especially of the zinc(II) carboxylates. This is because the prior art often employs metal compounds having problematic toxicological properties (for example Sn, Pb, etc.).

The invention has the further advantage that it can be used to produce PU or PIR foams, preferably rigid PU or PIR foams, having few foam defects.

The invention has the further advantage that high active concentrations of zinc salts in the reaction mixture are achievable. Better dissolution of the zinc salt may be effected, with the result that for example the altogether employable amount of a zinc-containing preparation added to a reaction mixture can actually be reduced.

The composition according to the invention preferably comprises at least one zinc(II) carboxylate, preferably selected from the group consisting of zinc(II) acetate, zinc(II) propionate, zinc(II) pivalate, zinc(II) 2-ethylhexanoate (zinc(II) octoate), zinc(II) isononanoate (zinc(II) 3,5,5-trimethylhexanoate), zinc(II) neodecanoate, zinc(II) ricinoleate, zinc(II) palmitate, zinc(II) stearate, zinc(II) oleate, zinc(II) laurate, zinc(II) naphthenate, zinc(II) benzoate, zinc(II) lactate, zinc(II) glycinate, zinc(II) hippurate, zinc(II) citrate and zinc(II) soaps, wherein the use of zinc(II) acetate and/or zinc(II) ricinoleate is very particularly preferred. Preferably employable zinc(II) soaps are zinc oleate, zinc palmitate and/or zinc stearate.

The zinc(II) carboxylate present in the composition according to the invention is employed in stoichiometric form, i.e. with Zn(II) and carboxylate in a molar ratio of 1 to 2. This means that no excess of (carboxylate or) carboxylic acid is employed or present. In the industrial production processes of zinc salts, the parent acids are sometimes employed in excess, with the result that the respective end product still contains an excess of the acid. Such an excess of the acid is not advantageous for the present invention.

The at least one zinc(II) carboxylate present in the composition according to the invention may be introduced into the relevant composition in any known manner but it is preferable to introduce the zinc(II) carboxylate in dissolved form, preferably using at least one nitrogen-containing compound V and optionally at least one carrier medium (i.e. solvent). In principle, any substances suitable as solvents may optionally be employed as carrier media. Glycols, alkoxylates and/or oils of synthetic and/or natural origin for example may preferably be optionally employed. Protic or aprotic solvents may preferably be optionally employed.

It is very particularly preferable when the at least one zinc(II) carboxylate present in the composition according to the invention is introduced into the composition in the form of a zinc(II) carboxylate-containing preparation, wherein this preparation preferably comprises at least one nitrogen-containing compound V according to the invention and optionally at least one carrier medium. A corresponding particularly preferably employable zinc(II) carboxylate-containing preparation is more particularly described hereinbelow.

The composition according to the invention contains at least one nitrogen-containing compound V, preferably selected from the group consisting of

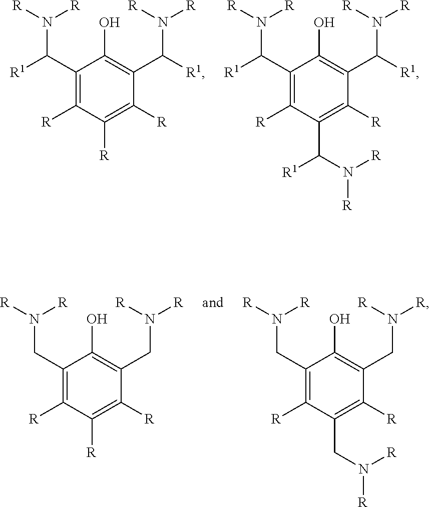

wherein
R=independently at each occurrence H or linear, branched or cyclic hydrocarbon radical having 1 to 20 carbon atoms which may be saturated, unsaturated or aromatic, optionally containing heteroatoms such as O or N,
R¹=independently at each occurrence H or linear, branched or cyclic hydrocarbon radical having 1 to 20 carbon atoms which may be saturated, unsaturated or aromatic.

Corresponding nitrogen-containing compounds V are known per se and are obtainable for example by reaction of optionally modified phenols with aldehydes and amines.

The at least one nitrogen-containing compound V may by preference be produced by reaction of phenol with aldehyde, preferably aldehyde having 1 to 20 carbon atoms, preferably formaldehyde, acetaldehyde, propionaldehyde, butyraldehyde, benzaldehyde, cinnamaldehyde, trimethylhexanal, pelargonaldehyde, acrolein and/or furfural and amine in the context of a Mannich reaction, wherein the starting material employed may also be selected from modified phenols bearing at least one substituent on the aromatic core, such as preferably cresol, xylenols, propylphenols, styrylphenols, alkylphenols and/or cardanol, wherein resorcinol and/or catechol-based structures are likewise preferably employable as modified phenols,

and the at least one nitrogen-containing compound V may preferably be produced by reaction of phenol with formaldehyde and at least one primary or secondary amine, preferably selected from the group consisting of dimethylamine, diethylamine, dipropylamine, diisopropylamine, dibutylamine, ethanolamine, diethanolamine, trimethylethanediamine, isophoronediamine, dimethylpropylamine, diethylenetriamine, triethylenetetramine, methylhydroxyethylamine, dimethylaminopropylamine, pyrrolidine, pyrrole, morpholine, piperazine, N,N-dimethyl-2-(2-methylaminoethoxy)ethan-1-amine, N-[2-[2-(dimethylamino)ethoxy]ethyl]-N-methyl-1,3-propanediamine, diisopropyldimethyldiethylenetriamine, bis(dimethylaminopropyl)amine, trimethylaminoethylethanolamine, N,N′-diisopropyl-N-methyl-bis(aminoethyl) ether and N,N′-diisobutyl-N-methyl-bis(aminoethyl) ether, wherein the starting material employed may in turn also be selected from modified phenols.

It is particularly preferable when the at least one nitrogen-containing compound V is selected from the group consisting of

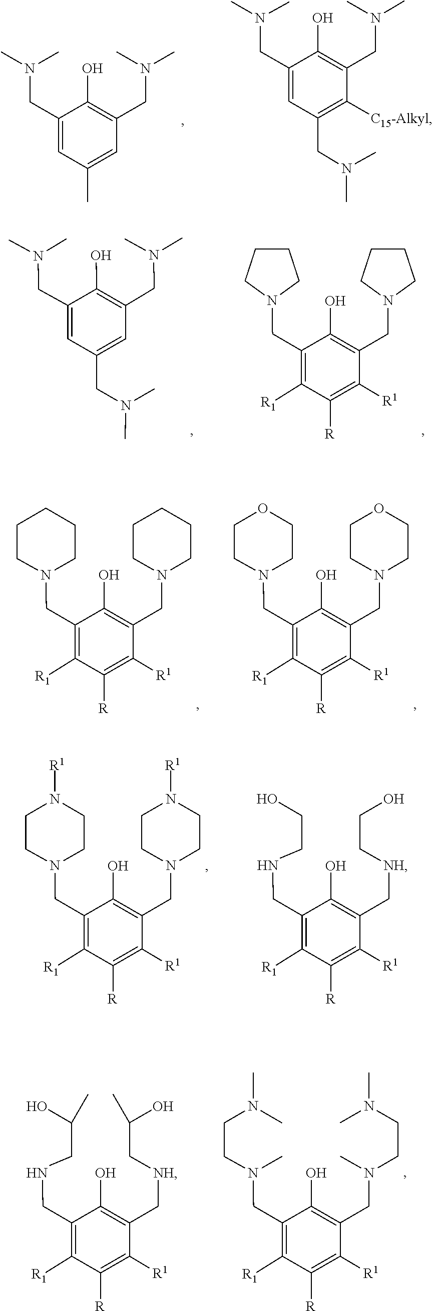

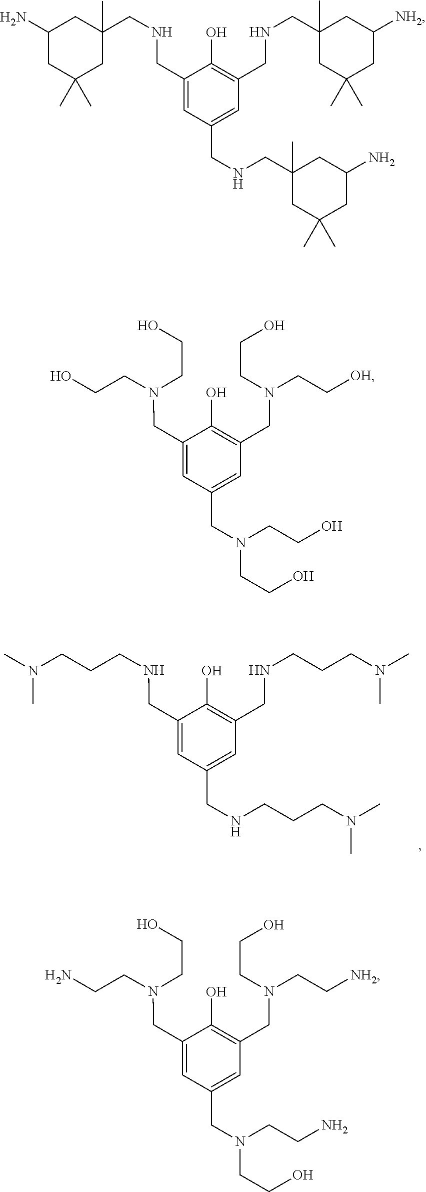

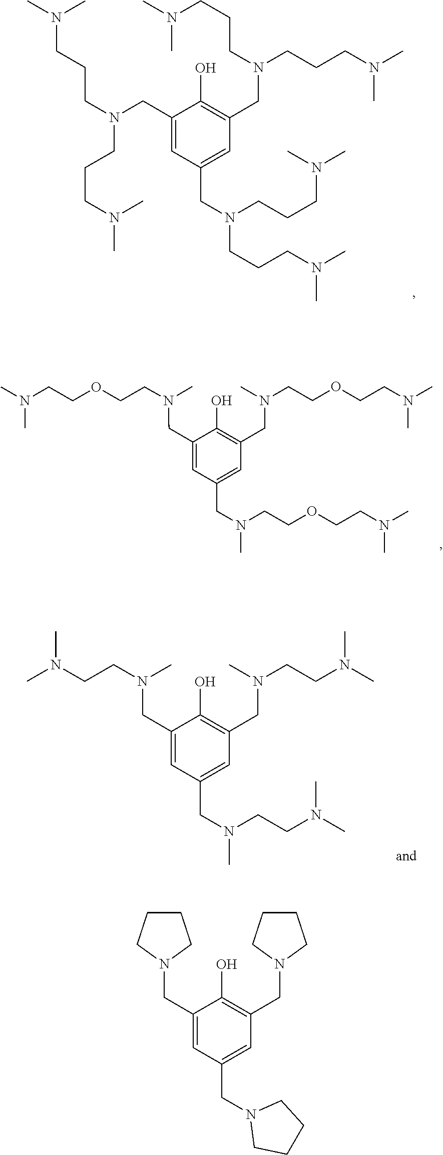

wherein
R=independently at each occurrence H or linear, branched or cyclic hydrocarbon radical having 1 to 20 carbon atoms which may be saturated, unsaturated or aromatic, optionally containing heteroatoms such as O or N,
R¹=independently at each occurrence H or linear, branched or cyclic hydrocarbon radical having 1 to 20 carbon atoms which may be saturated, unsaturated or aromatic,
   wherein the use of

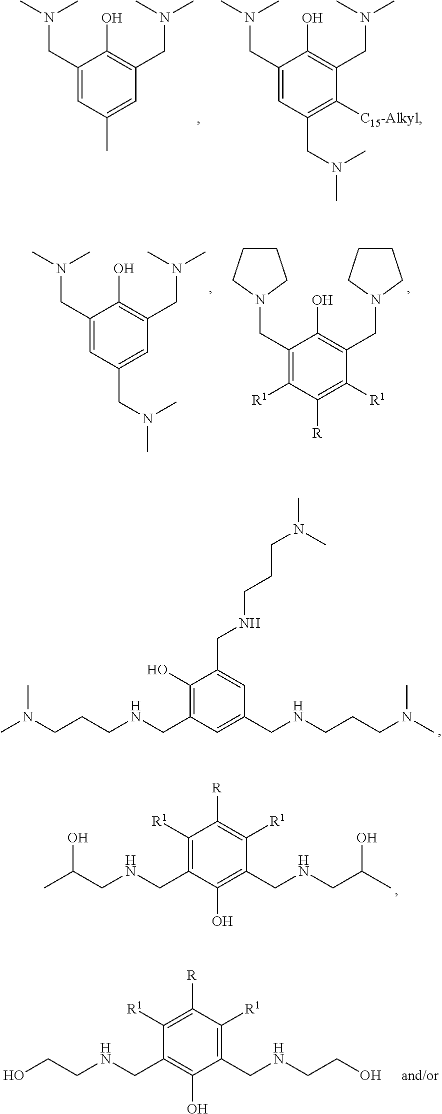

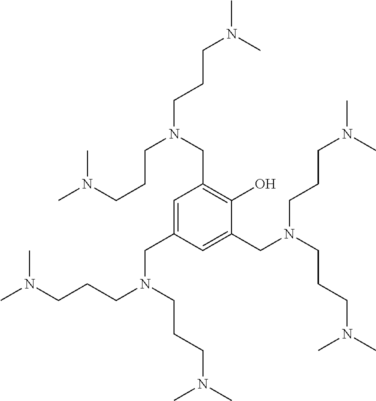

wherein
R=independently at each occurrence H or linear, branched or cyclic hydrocarbon radical having 1 to 20 carbon atoms which may be saturated, unsaturated or aromatic, optionally containing heteroatoms such as O or N,
R¹=independently at each occurrence H or linear, branched or cyclic hydrocarbon radical having 1 to 20 carbon atoms which may be saturated, unsaturated or aromatic,
   is very particularly preferred.

Greatest preference is given to the use of 2,6-bis[(dimethylamino)methyl]-4-methylphenol, 2,4,6-tris[(dimethylamino)methyl]cardanol, 2,4,6-tris[(dimethylamino)methyl]phenol, 2,6-bis[(dimethylamino)methyl]cardanol, 2,4,6-tris[(hydroxyethylamino)methyl]phenol, 2,4,6-tris[(hydroxypropylamino)methyl]phenol and/or 2,4,6-tris[(dimethylaminopropylamino)methyl]phenol.

The at least one nitrogen-containing compound V is preferably present in the composition according to the invention in a total amount of 1% to 80% by weight, preferably 2% to 60% by weight, wherein the % by weight values are in each case based on the total composition according to the invention.

It is preferable when the composition according to the invention contains altogether present zinc(II) carboxylate and altogether present nitrogen-containing compound V in a quantity ratio of 1:0.5 to 1:5 parts by weight relative to one another.

In the context of the present invention it is very particularly preferable to introduce the at least one zinc(II) carboxylate for use in PU or PIR reaction mixtures in dissolved form (i.e. liquid form at 25° C. and standard pressure) using at least one carrier medium.

It is preferably also possible to select a combination of at least one zinc(II) carboxylate and at least one nitrogen-containing compound V to ensure introduction in dissolved form, wherein this combination itself may preferably already have a sufficiently low viscosity and lack a propensity for crystallization.

The use of a carrier medium is optional. However, it is preferable to add the at least one zinc(II) carboxylate according to the invention to the reaction mixture in at least one carrier medium.

While the use of carrier media is optional it may be advantageous and preferable because it may contribute to the ability to establish desired viscosities for example and/or to reduce any propensity for crystallization.

It is particularly preferable when the composition according to the invention additionally contains at least one additional trimerization catalyst, by preference selected from carboxylates of ammonium, potassium and/or other alkali metals or alkaline earth metals, preferably selected from potassium carboxylates and carboxylates of ammonium cations, very particularly preferably selected from the group consisting of potassium acetate, potassium formate, potassium propionate, potassium butanoate, potassium pentanoate, potassium hexanoate, potassium heptanoate, potassium 2-ethylhexanoate, potassium pivalate, potassium octoate, potassium butyrate, potassium isobutyrate, potassium nonanoate, potassium decanoate, potassium ricinoleate, potassium stearate, potassium neodecanoate and carboxylates of tetramethylammonium, tetraethylammonium, triethylmethylammonium, tetrapropylammonium, tetrabutylammonium, dimethyldiallylammonium, trimethyl-(2-hydroxypropyl)ammonium, triethyl-(2-hydroxypropyl)ammonium, tripropyl-(2-hydroxypropyl)ammonium, tributyl-(2-hydroxypropyl)ammonium, trimethyl-(2-hydroxyethyl)ammonium, triethyl-(2-hydroxyethyl)ammonium, tripropyl-(2-hydroxyethyl)ammonium, tributyl-(2-hydroxyethyl)ammonium, dimethylbenzyl-(2-hydroxyethyl)ammonium and/or dimethylbenzyl-(2-hydroxypropyl)ammonium, wherein the carboxylates are preferably acetates, propionates, butanoates, pentanoates, pivalates, octoates, nonanoates, decanoates, neodecanoates, ricinoleates and/or stearates.

The additional trimerization catalysts as such do not themselves contain any zinc.

The at least one additional trimerization catalyst makes it possible to adapt the reaction rate to the preferred level if desired. The at least one additional trimerization catalyst may also be a constituent of a zinc(II) carboxylate-containing preparation. However, it may also be supplied to the composition according to the invention separately, i.e. separate from zinc(II) carboxylate(s).

A preferred composition according to the invention comprises the at least one additional trimerization catalyst in a total amount of 10% to 90% by weight, preferably of 20% to 80% by weight, based on the total composition according to the invention.

It is preferable when optionally (preferably compulsorily) present additional trimerization catalyst is introduced into the composition according to the invention separately from the at least one zinc(II) carboxylate.

It is likewise preferable when the composition according to the invention is free from Sb carboxylate and/or Sn carboxylate.

As already mentioned above, the composition according to the invention contains at least one tertiary amine of formula (X)

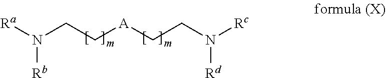

where
m is independently at each occurrence 1 or 2,
A is O, S or N—R^{e},
R^{a}, R^{b}, R^{c}, R^{d} and R^{e} are each independently of one another identical or different linear, branched or cyclic alkyl radicals having 1 to 20 carbon atoms.

It is particularly preferable when the at least one tertiary amine of formula (X) is selected from the group consisting of pentamethyldiethylenetriamine, bis(2-dimethylaminoethyl) ether, tris(dimethylaminopropyl)amine, N-[2-[2-(dimethylamino)ethoxy]ethyl]-N-methyl-1,3-propanediamine, diisopropyltrimethyldiethylenetriamine, bis(dimethylaminopropyl)methylamine, trimethylaminoethylethanolamine, bis(2-isopropylmethylaminoethyl) ether, bis(2-isobutylmethylaminoethyl) ether, N,N′-diisopropyl-N,N′-dimethyl-bis(aminoethyl) ether, N,N,N′-triisopropyl-N′-methyl-bis(aminoethyl) ether and N,N′-diisobutyl-N,N′-dimethyl-bis(aminoethyl) ether.

The at least one tertiary amine of formula (X), preferably pentamethyldiethylenetriamine, bis(2-dimethylaminoethyl) ether, tris(dimethylaminopropyl)amine, N-[2-[2-(dimethylamino)ethoxy]ethyl]-N-methylpropane-1,3-diamine, 2-[[2-[2-(dimethylamino)ethoxy]ethyl]methylamino]ethanol, diisopropyltrimethyldiethylenetriamine, bis(dimethylaminopropyl)methylamine and/or trimethylaminoethylethanolamine, shall especially assume the function of catalyst.

A preferred composition according to the invention comprises at least one tertiary amine of formula (X), preferably pentamethyldiethylenetriamine, bis(2-dimethylaminoethyl) ether, tris(dimethylaminopropyl)amine, N-[2-[2-(dimethylamino)ethoxy]ethyl]-N-methylpropane-1,3-diamine, 2-[[2-[2-(dimethylamino)ethoxy]ethyl]methylamino]ethanol, diisopropyltrimethyldiethylenetriamine, bis(dimethylaminopropyl)methylamine and/or trimethylaminoethylethanolamine, in a total amount of >0% to 20% by weight, preferably of 1% to 15% by weight, based on the total composition according to the invention.

That is to say a particularly preferred composition according to the invention comprises a polyisocyanate component, a polyol component, at least one zinc(II) carboxylate, at least one tertiary amine of formula (X), at least one nitrogen-containing compound V and at least one additional trimerization catalyst.

In a preferred embodiment of the invention the total mass fraction of altogether employed zinc(II) carboxylate in the finished polyurethane or polyisocyanurate foam is preferably from 0.01% to 10% by weight, preferably from 0.1% to 5% by weight.

It is preferable when the composition according to the invention comprises water and/or at least one blowing agent, optionally at least one flame retardant and/or further additives that are advantageously employable in the production of polyurethane or polyisocyanurate foam. Further catalysts may also be present in addition to the zinc(II) carboxylate.

A particularly preferred composition according to the invention contains the following constituents:
a) a polyisocyanate component,
b) a polyol component,
c) at least one zinc(II) carboxylate with the proviso that the zinc(II) carboxylate present is employed in stoichiometric form, i.e. with Zn(II) and carboxylate in a molar ratio of 1 to 2,
d) at least one tertiary amine of formula (X)

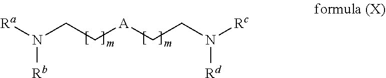

where
m is independently at each occurrence 1 or 2,
A is O, S or N—R^{e},
R^{a}, R^{b}, R^{c}, R^{d} and R^{e} are each independently of one another identical or different linear, branched or cyclic alkyl radicals having 1 to 20 carbon atoms,
e) at least one foam stabilizer,
f) at least one blowing agent,
g) at least one nitrogen-containing compound V,
h) at least one additional trimerization catalyst,
i) optionally further additives, preferably fillers and/or flame retardants.

As has already been made apparent the at least one zinc(II) carboxylate may be introduced into the composition according to the invention in different ways, for example in pure form, dissolved form or mixed with other constituents.

It is particularly preferable to introduce the at least one zinc(II) carboxylate into the composition according to the invention not in pure form but rather in the form of a zinc(II) carboxylate-containing preparation, wherein the preparation preferably additionally comprises at least one nitrogen-containing compound V and may optionally contain at least one carrier medium.

The present invention thus further provides a zinc(II) carboxylate-containing preparation suitable for catalysis in the production of PU or PIR foam which comprises:
i) at least one zinc(II) carboxylate in a total amount of 2% to 50% by weight, preferably 5% to 45% by weight, particularly preferably 10% to 40% by weight, with the proviso that the zinc(II) carboxylate present is employed in stoichiometric form, i.e. with Zn(II) and carboxylate in a molar ratio of 1 to 2,
ii) optionally at least one carrier medium in a total amount of 0% to 95% by weight, preferably 10% to 90% by weight, particularly preferably 20% to 70% by weight,
iii) at least one nitrogen-containing compound V which comprises at least two N atoms and which is a modified phenol in a total amount of 1% to 90% by weight, preferably 2% to 60% by weight, particularly preferably 5% to 50% by weight, in each case based on the total preparation, wherein the components (i) to (iii) must together account for by preference at least 51% by weight, preferably at least 70% by weight, particularly preferably at least 80% by weight, of the total preparation, and preferably further comprises
iv) at least one tertiary amine as defined in embodiment 1 or 7 in amounts of 1% to 30% by weight, preferably 2% to 25% by weight, particularly preferably 5% to 20% by weight, based on the total preparation,
v) optionally at least one additional trimerization catalyst, preferably as defined in embodiment 8, in amounts of 1% to 90% by weight, preferably 2% to 60% by weight, particularly preferably 5% to 50% by weight, in turn in each case based on the total preparation, wherein the components i) to v) must altogether account for preferably at least 52% by weight of the total preparation.

The zinc-containing preparations according to the invention make it possible to achieve a higher zinc concentration and/or a lower viscosity than is known in the prior art based on the respective preparation.

It is preferable when the zinc(II) carboxylate-containing preparation comprises no additional trimerization catalyst. In an alternative embodiment it may be preferable when the zinc(II) carboxylate-containing preparation comprises as a further constituent v) at least one additional trimerization catalyst, preferably as defined in embodiment 8, preferably in amounts of 1% to 90% by weight, more preferably 2% to 60% by weight, particularly preferably 5% to 50% by weight, in turn in each case based on the total preparation,

wherein the components i) to v) must together account for by preference at least 52% by weight, preferably at least 80% by weight, particularly preferably at least 90% by weight, of the total preparation.

It is preferable to employ at least one carrier medium to provide a preferred zinc(II) carboxylate-containing preparation which is particularly uncomplicated to employ. Preference would here be given for example to a mixture or solution which exhibits little or no turbidity, i.e. preferably no emulsions, suspensions or dispersions which can suffer problems such as phase separation or sedimentation for example.

A lowest possible viscosity would likewise be preferable, so that the preparation does not make any special demands of pumps or other technical apparatuses for processing for example. Preferred viscosities are less than 10 Pa-s, preferably less than 8 Pa-s, particularly preferably less than 6 Pa-s, measured with a Brookfield viscometer at 25° C.

The invention further provides a process for producing polyurethane or polyisocyanurate foam by reacting a polyol component with a polyisocyanate component, characterized in that it is carried out using a composition according to the invention as described above, preferably according to any of embodiments 1 to 10, or using a zinc(II) carboxylate-containing preparation according to the invention, preferably according to embodiment 11,

wherein when using a zinc(II) carboxylate-containing preparation it is preferable when the total mass fraction of zinc(II) carboxylate-containing preparation in the finished polyurethane foam is 0.05% to 10% by weight, preferably from 0.2% to 5% by weight.

In addition to the preferably employable zinc(II) carboxylate-containing preparation according to the invention, it is also possible to employ further catalysts, very particularly preferably at least one tertiary amine of formula (X) as described above. The additional use of at least one trimerization catalyst as described above is likewise preferred.

It is preferable when the zinc(II) carboxylate-containing preparation comprises at least one carrier medium, wherein the zinc(II) carboxylate-containing preparation is particularly preferably supplied to the reaction mixture for production of the polyurethane or polyisocyanurate foam, preferably rigid PU or PIR foam, in a carrier medium preferably comprising glycols, alkoxylates and/or oils of synthetic and/or natural origin.

The invention further provides a polyurethane or polyisocyanurate foam, preferably rigid polyurethane or polyisocyanurate foam, obtainable by the process according to the invention as described above.

The invention further provides for the use of a composition according to any of embodiments 1 to 10 or a preparation according to embodiment 11 in the production of polyurethane or polyisocyanurate foams, preferably rigid polyurethane or polyisocyanurate foams, preferably to improve the performance characteristics of the polyurethane or polyisocyanurate foam, preferably rigid polyurethane or polyisocyanurate foam, in particular to increase the compressive strength of the polyurethane or polyisocyanurate foam, preferably rigid polyurethane or polyisocyanurate foam, at an early juncture compared to polyurethane or polyisocyanurate foams, preferably rigid polyurethane or polyisocyanurate foams, produced without zinc(II) carboxylate, wherein compressive strength is determinable according to DIN EN ISO 844:2014-11. The expression “early point in time” is preferably understood to mean a point in time in the range from 3 minutes to 10:30 minutes, preferably 8 minutes, after initiation of the polymerization, preferably after initiation of the polymerization by addition of the polyisocyanate component.

In addition, the present invention further provides for the use of polyurethane or polyisocyanurate foams, preferably rigid polyurethane or polyisocyanurate foams, according to the invention for thermal insulation purposes, preferably as insulating panels and/or insulation material, and also for refrigeration equipment comprising a polyurethane or polyisocyanurate foam, preferably a rigid polyurethane or polyisocyanurate foam, according to the invention as insulating material.

Individual preferably employable components will now be more particularly described.

Polyols suitable as the polyol component in the context of the present invention include all organic compounds having at least two isocyanate-reactive groups, preferably OH groups, and also preparations thereof. The polyol component especially comprises at least one organic compound containing at least two hydroxyl groups (—OH). Mixtures of at least two suitable polyols may preferably be employed.

Preferred polyols include all polyether polyols and/or polyester polyols and/or hydroxyl-containing aliphatic polycarbonates, in particular polyether polycarbonate polyols, and/or polyols of natural origin, so-called “natural oil-based polyols” (NOPs), typically employable for producing polyurethane systems, especially polyurethane coatings, polyurethane elastomers or foams. The polyols preferably have a functionality of 1.8 to 8 and number-average molecular weights preferably in the range from 500 to 15 000. It is preferable to employ polyols having OH numbers in the range from 10 to 1200 mg KOH/g.

Polyether polyols are preferably employable. These are obtainable by known processes, for example by anionic polymerization of alkylene oxides in the presence of alkali metal hydroxides, alkali metal alkoxides or amines as catalysts and by addition of at least one starter molecule which preferably contains 2 or 3 reactive hydrogen atoms in bonded form, or by cationic polymerization of alkylene oxides in the presence of Lewis acids, for example antimony pentachloride or boron trifluoride etherate, or by double metal cyanide catalysis. Suitable alkylene oxides contain 2 to 4 carbon atoms in the alkylene radical. Examples are tetrahydrofuran, 1,3-propylene oxide and 1,2- or 2,3-butylene oxide; preference is given to using ethylene oxide and 1,2-propylene oxide. The alkylene oxides may be used individually, cumulatively, in blocks, in alternating succession or as mixtures. Starter molecules used may in particular be compounds having at least 2, preferably 2 to 8, hydroxyl groups, or having at least two primary amino groups in the molecule. Starter molecules used may, for example, be water, di-, tri- or tetrahydric alcohols such as ethylene glycol, propane-1,2- and -1,3-diol, diethylene glycol, dipropylene glycol, glycerol, trimethylolpropane, pentaerythritol, castor oil, etc., higher polyfunctional polyols, especially sugar compounds, for example glucose, sorbitol, mannitol and sucrose, polyhydric phenols, resols, for example oligomeric condensation products of phenol and formaldehyde and Mannich condensates of phenols, formaldehyde and dialkanolamines, and also melamine, or amines such as aniline, EDA, TDA, MDA and PMDA, more preferably TDA and PMDA. The choice of the suitable starter molecule is dependent on the respective field of application of the resulting polyether polyol in the production of polyurethane.

Polyester polyols are preferably employable. These are based on esters of polybasic aliphatic or aromatic carboxylic acids, preferably having 2 to 12 carbon atoms. Examples of aliphatic carboxylic acids are succinic acid, glutaric acid, adipic acid, suberic acid, azelaic acid, sebacic acid, decanedicarboxylic acid, maleic acid and fumaric acid. Examples of aromatic carboxylic acids are phthalic acid, isophthalic acid, terephthalic acid and the isomeric naphthalenedicarboxylic acids. The polyester polyols are obtained by condensation of these polybasic carboxylic acids with polyhydric alcohols, preferably diols or triols having 2 to 12, particularly preferably having 2 to 6, carbon atoms, preferably trimethylolpropane and glycerol.

Polyether carbonate polyols are preferably employable. These are polyols containing carbon dioxide in the bonded form of the carbonate. Since carbon dioxide is formed in large amounts as a by-product in many processes in the chemical industry, the use of carbon dioxide as comonomer in alkylene oxide polymerizations is of particular interest from a commercial viewpoint. Partial replacement of alkylene oxides in polyols with carbon dioxide has the potential to distinctly lower costs for the production of polyols. Moreover, the use of CO₂ as comonomer is environmentally very advantageous, since this reaction constitutes the conversion of a greenhouse gas into a polymer.

The preparation of polyether polycarbonate polyols by addition of alkylene oxides and carbon dioxide onto H-functional starter substances with the use of catalysts has long been known. Various catalyst systems may be used here: The first generation was that of heterogeneous zinc or aluminium salts, as described, for example, in U.S. Pat. No. 3,900,424 or U.S. Pat. No. 3,953,383. Mono- and binuclear metal complexes have also been successfully employed for copolymerization of CO2 and alkylene oxides (cf. for example WO 2010/028362, WO 2009/130470, WO 2013/022932 or WO 2011/163133). The most important class of catalyst systems for the copolymerization of carbon dioxide and alkylene oxides is that of double metal cyanide catalysts, also referred to as DMC catalysts (cf. for example U.S. Pat. No. 4,500,704, WO 2008/058913). Suitable alkylene oxides and H-functional starter substances are those also used for preparing carbonate-free polyether polyols, as described above.

Polyols based on renewable raw materials, “natural oil-based polyols” (NOPs), are preferably usable. NOPs for production of polyurethane foams are of increasing interest with regard to the limited availability in the long term of fossil resources, namely oil, coal and gas, and against the background of rising crude oil prices, and have already been described many times in such applications (cf. for example WO 2005/033167; US 2006/0293400, WO 2006/094227, WO 2004/096882, US 2002/0103091, WO 2006/116456 and EP 1678232). A number of such polyols are now available on the market from various manufacturers (cf. for example WO2004/020497, US2006/0229375, WO2009/058367). Depending on the base raw material (e.g. soybean oil, palm oil or castor oil) and subsequent processing, polyols having different profiles of properties are obtained. A distinction may essentially be made between two groups: a) polyols based on renewable raw materials that are modified such that they may be used to an extent of 100% in the production of polyurethanes (cf. for example WO2004/020497, US2006/0229375); b) polyols based on renewable raw materials that on account of their processing and properties are able to replace the petrochemical-based polyol only up to a certain proportion (cf. for example WO 2009/058367).

A further class of preferably employable polyols is that of the so-called filled polyols (polymer polyols). The characteristic feature of these is that they contain dispersed solid organic fillers preferably up to a solids content of 40% or more. Usable polyols include SAN, PUD and PIPA polyols. SAN polyols are highly reactive polyols containing a dispersed copolymer based on styrene-acrylonitrile (SAN). PUD polyols are highly reactive polyols containing polyurea, likewise in dispersed form. PIPA polyols are highly reactive polyols containing a dispersed polyurethane, for example formed by in situ reaction of an isocyanate with an alkanolamine in a conventional polyol.

Polyols having a molar mass of less than 1000 g/mol are preferably employable. Preference is further given to polyols having a functionality of less than 3. It is in particular preferable not to use triols having molar masses exceeding 1000 g/mol. Each of these is a particularly preferred form of the invention.

A preferred ratio of isocyanate and polyol, expressed as the index of the formulation, i.e. as the stoichiometric ratio of isocyanate groups to isocyanate-reactive groups (e.g. OH groups, NH groups) multiplied by 100, is in the range from 10 to 1000, preferably 40 to 700, more preferably 60 to 600, more preferably 150 to 550, yet more preferably 250 to 500, very particularly preferably 300 to 450. An index of 100 represents a molar ratio of reactive groups of 1:1.

Preference is given to PIR formulations based on at least 70%, 80% or 90% by weight of polyester in the polyol component.

In a particularly preferred embodiment polyester polyols based on aromatic carboxylic acids are employed in amounts of more than 50 parts by mass, preferably more than 70 parts by mass, based on 100 parts by mass of the total polyol component.

Preferred aromatic polyester polyols have OH numbers in the range from 150 to 400 mg KOH/g, preferably 170 to 350, very particularly preferably 180 to 300 mg KOH/g.

The polyisocyanate component employed is preferably at least one organic polyisocyanate having at least two isocyanate functions.

Suitable polyisocyanates in the context of the present invention include all isocyanates containing at least two isocyanate groups. It is preferably possible to employ any aliphatic, cycloaliphatic, arylaliphatic and preferably aromatic polyfunctional isocyanates known per se. It is particularly preferable to employ isocyanates in a range from 60 to 200 mol % relative to the sum of isocyanate-consuming components.

It is preferable to employ mixtures of at least two suitable polyisocyanates.

Examples include alkylene diisocyanates having 4 to 12 carbon atoms in the alkylene radical, for example dodecane 1,12-diisocyanate, 2-ethyltetramethylene 1,4-diisocyanate, 2-methylpentamethylene 1,5-diisocyanate, tetramethylene 1,4-diisocyanate and preferably hexamethylene 1,6-diisocyanate (HMDI), cycloaliphatic diisocyanates such as cyclohexane 1,3- and 1,4-diisocyanate and also any mixtures of these isomers, 1-isocyanato-3,3,5-trimethyl-5-isocyanatomethylcyclohexane (isophorone diisocyanate or IPDI for short), hexahydrotolylene 2,4- and 2,6-diisocyanate and also the corresponding isomeric mixtures, and preferably aromatic diisocyanates and polyisocyanates such as toluene 2,4- and 2,6-diisocyanate (TDI) and the corresponding isomeric mixtures, naphthalene diisocyanate, diethyltoluene diisocyanate, mixtures of diphenylmethane 2,4′- and 2,2′-diisocyanates (MDI) and polyphenyl polymethylene polyisocyanates (crude MDI) and mixtures of crude MDI and toluene diisocyanates (TDI). The organic diisocyanates and polyisocyanates may be used individually or in the form of mixtures thereof. It is likewise possible to use corresponding “oligomers” of the diisocyanates (for example IPDI trimer based on isocyanurate, biuret and/or uretdione formation). In addition, the use of prepolymers based on the abovementioned isocyanates is possible.

It is also possible to use isocyanates which have been modified by the incorporation of urethane, uretdione, isocyanurate, allophanate and other groups, called modified isocyanates.

Organic polyisocyanates that are particularly suitable and therefore employed with particular preference are various isomers of tolylene diisocyanate (tolylene 2,4- and 2,6-diisocyanate (TDI), in pure form or as isomer mixtures of varying composition), diphenylmethane 4,4′-diisocyanate (MDI), “crude MDI” or “polymeric MDI” (comprising the 4,4′ isomer and also the 2,4′ and 2,2′ isomers of MDI and products having more than two rings) and also the two-ring product referred to as “pure MDI” that is composed predominantly of 2,4′ and 4,4′ isomer mixtures, and prepolymers derived therefrom. Examples of particularly suitable isocyanates are detailed, for example, in EP 1712578, EP 1161474, WO 00/58383, US 2007/0072951, EP 1678232 and WO 2005/085310, which are hereby fully incorporated by reference.

Optional catalysts may be used in addition to the catalyst according to the invention, i.e. the at least one zinc(II) carboxylate as described above. Possible catalysts may for example also include the at least one tertiary amine of formula (X) and for example the at least one additional trimerization catalyst as described above.

Suitable, preferably suitable additional optional, catalysts in the context of the present invention include any compounds capable of accelerating the reaction of isocyanates with OH functions, NH functions or other isocyanate-reactive groups and with isocyanates themselves. It is possible here to make use of the customary catalysts known from the prior art, including, for example, amines (cyclic, acyclic; monoamines, diamines, oligomers having one or more amino groups), ammonium compounds, organometallic compounds and metal salts, preferably those of potassium, tin, iron, bismuth. In particular, mixtures of two or more components may be employed as catalysts.

Optional foam stabilizers employed may also include substances known from the prior art, preferably Si-free surfactants or else organomodified siloxanes.

The use of such substances in the production of PU or PIR foams is known. In the context of the present invention it is possible to optionally employ any compounds which assist foam production (stabilization, cell regulation, cell opening etc.). These compounds are well known from the prior art.

Corresponding siloxanes usable in the context of this invention are described, for example, in the following patent specifications: CN 103665385, CN 103657518, CN 103055759, CN 103044687, US 2008/0125503, US 2015/0057384, EP 1520870 A1, EP 1211279, EP 0867464, EP 0867465, EP 0275563. The abovementioned documents are hereby incorporated by reference and are considered to form part of the disclosure content of the present invention. The use of polyether-modified siloxanes is particularly preferred.

The use of blowing agents is optional, depending on which foaming process is used. It is possible to employ chemical and physical blowing agents. The choice of blowing agent is strongly dependent on the nature of the system.

In a particularly preferred embodiment no HFOs are used as blowing agent.

Depending on the amount of blowing agent used, a foam having high or low density can be produced. This makes it possible to produce foams having for example densities of 5 kg/m³ to 900 kg/m³. Preferred densities are 8 to 800 kg/m³, particularly preferably 10 to 600 kg/m³, in particular 30 to 150 kg/m³.

Physical blowing agents used may be corresponding compounds having appropriate boiling points. It is likewise possible to use chemical blowing agents which react with NCO groups to liberate gases, for example water or formic acid. Examples of blowing agents include liquefied CO2, nitrogen, air, volatile liquids, for example hydrocarbons having 3, 4 or 5 carbon atoms, preferably cyclopentane, isopentane and/or n-pentane, hydrofluorocarbons, preferably HFC 245fa, HFC 134a and/or HFC 365mfc, hydrochlorofluorocarbons, preferably HCFC 141b, hydrofluoroolefins (HFOs) and/or hydrohaloolefins such as for example 1234ze, 1234yf, 1233zd(E) or 1336mzz, oxygen-containing compounds such as methyl formate, acetone and/or dimethoxymethane, and/or hydrochlorocarbons, preferably dichloromethane and/or 1,2-dichloroethane.

Suitable water contents in the context of the present invention depend on whether or not one or more blowing agents are used in addition to the water. In the case of purely water-blown foams the values are preferably 1 to 20 pphp; when other blowing agents are additionally used the amount of water used is reduced to preferably 0.1 to 5 pphp. The abbreviation pphp stands for parts per hundred parts polyol, i.e. parts by weight per 100 parts by weight of polyol. This is a method for reporting amounts of components of a foam formulation which is typical in industry.

Further optional additives that may be used preferably include all substances which are known from the prior art and are used in the production of polyurethanes, especially of polyurethane or PIR foams, for example crosslinkers and chain extenders, stabilizers against oxidative degradation (so-called antioxidants), flame retardants, surfactants, biocides, cell-refining additives, cell openers, solid fillers, antistatic additives, nucleating agents, thickeners, dyes, pigments, colour pastes, fragrances, and emulsifiers, etc.

The process according to the invention for producing PU or PIR foams, preferably rigid PU or PIR foams, may be performed by the known methods, for example by manual mixing or preferably using foaming machines. If the process is conducted using foaming machines, it is possible to use high-pressure or low-pressure machines. The process according to the invention can be carried out either batchwise or continuously.

A preferred polyurethane or polyisocyanurate foam formulation in the context of the present invention results in a foam density of 5 to 900 kg/m3 and preferably has the composition shown in Table 1.

TABLE 1
Composition of a preferred polyurethane
or polyisocyanurate foam formulation

| Component | Weight fraction |
|---|---|
| Polyol | 0.1 to 100 |
| Amine catalyst comprising tertiary | >0 to 5 |
| amine of formula (X) | |
| Optional additional catalysts | 0 to 10 |
| Inventive zinc(II) carboxylate | 0.1 to 10 |
| Foam stabilizer (Si-free or Si-containing) | 0 to 5 |
| Water | 0.01 to 20 |
| Blowing agent | 0 to 40 |
| Further additives (flame retardants, etc.) | 0 to 90 |
| Nitrogen-containing compound V | >0 to 10 |
| Additional trimerization catalyst | >0 to 10 |
| Isocyanate index: 10 to 1000 | |

For further preferred embodiments and configurations of the process according to the invention, reference is also made to the details previously given above in connection with the composition according to the invention.

As mentioned above the invention further provides a PU or PIR foam, preferably rigid PU or PIR foam, obtainable by the aforementioned process.

Rigid PU or PIR foam is an established technical term. The known and fundamental difference between flexible foam and rigid foam is that flexible foam shows elastic behaviour and hence deformation is reversible. By contrast, rigid foam undergoes permanent deformation. In the context of the present invention rigid PU or PIR foam is especially understood to mean a foam according to DIN 7726:1982-05 that has a compressive strength according to DIN 53 421/DIN EN ISO 604:2003-12 of by preference 20 kPa, preferably 80 kPa, more preferably 100 kPa, yet more preferably 150 kPa, particularly preferably 180 kPa. In addition, the rigid PU or PIR foam advantageously has a closed-cell content according to DIN EN ISO 4590:2016-12 of greater than 50%, preferably greater than 80% and particularly preferably greater than 90%. Rigid PU or PIR foam is particularly preferable in the context of the entire present invention.

The polyurethane or PIR foam has a density of preferably 5 to 900 kg/m³, more preferably 8 to 800 kg/m³, particularly preferably 10 to 600 kg/m³, in particular 30 to 150 kg/m³.

It is preferably possible to produce predominantly closed-cell foams. The closed cell content is preferably >80%, preferably >90%.

The PU or PIR foams according to the invention may preferably be used as or for production of insulation materials, preferably insulating panels, refrigerators, insulating foams, headliners, packaging foams or spray foams.

The PU or PIR foams according to the invention are advantageously employable especially in the refrigerated warehouse, refrigeration equipment and domestic appliances industry, for example for production of insulating panels for roofs and walls, as insulating material in containers and warehouses for frozen goods, and for refrigeration and freezing equipment.

Further preferred fields of application are in vehicle construction, especially for production of vehicle headliners, body parts, interior trim, refrigerated vehicles, large containers, transport pallets, packaging laminates, in the furniture industry, for example for furniture parts, doors, claddings and in electronics applications.

PU or PIR foams (polyurethane or polyisocyanurate foams) may preferably be used as insulating material for refrigeration equipment.

The invention further provides for the use of the PU or PIR foam as insulation material in refrigeration technology, in refrigeration equipment, in the construction sector, automobile sector, shipbuilding sector and/or electronics sector, as insulating panels, as spray foam and as one-component foam.

The invention will now be more particularly described by way of example without limiting the invention in any way. Where ranges, general formulae or classes of compounds are reported, these are intended to encompass not only the corresponding ranges or groups of compounds explicitly mentioned but also all subranges and subgroups of compounds obtainable by selecting individual values (ranges) or compounds. Where documents are cited in the context of the present description, the entire content thereof, particularly with regard to the subject matter that forms the context in which the document has been cited, is intended to form part of the disclosure content of the present invention. Unless otherwise stated, reported percentages are percentages by weight. Where average values are reported these are weight-averages unless otherwise stated. Where parameters determined by measurement are reported the measurements have been performed at a temperature of 25° C. and standard pressure (preferably 101 325 Pa) unless otherwise stated.

### EXAMPLES

The following raw materials were employed:
Stepanpol® PS 2352: polyester polyol from Stepan
Daltolac@R 471: polyether polyol from Huntsman
TCPP: tris(2-chloroisopropyl) phosphate from ICL
KOSMOS® 75 from Evonik Operations GmbH, catalyst based on potassium octoate
KOSMOS® 33 MEG from Evonik Operations GmbH, catalyst based on potassium acetate
POLYCAT® 5 from Evonik Operations GmbH, amine catalyst
POLYCAT® DP from Evonik Operations GmbH, amine catalyst
POLYCAT® 9 from Evonik Operations GmbH, amine catalyst
POLYCAT® 206 from Evonik Operations GmbH, amine catalyst
POLYCAT® 77 from Evonik Operations GmbH, amine catalyst
TEGOAMIN® BDE from Evonik Operations GmbH, amine catalyst
DABCO® T from Evonik Operations GmbH, amine catalyst
DABCO® NE 300 from Evonik Operations GmbH, amine catalyst
Amine no. 1: composed of N,N′-diisopropyl-N,N′-dimethyl-bis(aminoethyl) ether, N,N,N′-triisopropyl-N′-methyl-bis(aminoethyl) ether as described in WO 2023/043980 A2 in example 4 as structure V and VI
Amine no. 2: composed of N,N′-diisobutyl-N,N′-dimethyl-bis(aminoethyl) ether as described in WO 2023/043980 A2 in example 8 as structure XVII
MDI (44V20): Desmodur® 44V20L from Covestro, diphenylmethane 4,4′-diisocyanate (MDI) with isomeric and higher-functionality homologues
Tegostab® B 8460 from Evonik Operations GmbH, foam-stabilizing surfactant

### Production of Zinc-Containing Preparations:

Various preparations are produced and can then be combined in the foaming operations to afford inventive (or noninventive) compositions.

The corresponding components may be added to the reaction mixture to be foamed in preformulated form or as individual components.

Inventive examples are those containing zinc.

### Raw Materials for Producing the Zinc-Containing Preparations:

Zn Ac: Zinc acetate dihydrate (obtainable from Sigma-Aldrich)
Zn Ric: Zinc ricinoleate (obtainable from Evonik as TEGODEO@PY 88 G)
EDA-PO: N,N,N′,N′-tetrakis(2-hydroxypropyl)ethylenediamine—(described in WO 2022/218657 A1 and obtainable from BASF)
V1: 2,4,6-tris[(dimethylamino)methyl]phenol (obtainable from Sigma-Aldrich)
DEG: Diethylene glycol.

### Production of the Preparations:

The liquid components such as DEG, EDA-PO or V1 were initially charged, the zinc salts Zn Ac or Zn Ric were subsequently added and the components were stirred at about 50° C. to obtain a clear mixture.

Table 1 summarizes the compositions of the preparations with the weight fractions of the individual components.

The viscosities were determined with a Brookfield viscometer at room temperature, 23° C.

TABLE 1
Overview of zinc-containing preparations (ZZ) A to L and their viscosities

| | | | parts | | parts | | parts | | |
|---|---|---|---|---|---|---|---|---|---|
| Name | | Zinc salt | by wt. | | by wt. | | by wt. | Viscosity | |
| A | inv. | Zn Ac | 40 | V1 | 60 | | | 200 | Pa · s |
| B | noninv. | Zn Ac | 40 | EDA-PO | 60 | | | >500 | Pa · s |
| C | inv. | Zn Ric | 70 | V1 | 30 | | | 60 | Pa · s |
| D | noninv. | Zn Ric | 70 | EDA-PO | 30 | | | >200 | Pa · s |
| E | inv. | Zn Ric | 56 | V1 | 12 | DEG | 32 | 7260 | mPa · s |
| F | inv. | Zn Ac | 30 | V1 | 40 | DEG | 30 | 2100 | mPa · s |
| G | noninv. | Zn Ac | 30 | EDA-PO | 40 | DEG | 30 | 8800 | mPa · s |
| H | inv. | Zn Ac | 40 | V1 | 30 | DEG | 30 | 3060 | mPa · s |
| J | inv. | Zn Ac | 45 | V1 | 30 | DEG | 25 | 7020 | mPa · s |
| K | inv. | Zn Ac | 30 | V1 | 20 | DEG | 50 | 390 | mPa · s |
| L | noninv. | Zn Ac | 30 | EDA-PO | 20 | DEG | 50 | 370 | mPa · s |

Preparations B, D, G and L are noninventive.
(inv. = inventive; noninv. = noninventive)

V1 made it possible to produce mixtures having higher zinc contents which did not crystallize out and had lower viscosities than were possible with EDA-PO.

Thus at 40% zinc acetate the viscosities with EDA-PO were greater than 500 Pa-s (composition B) and with V1 about 200 Pa-s (preparation A).

The same trend was apparent when using zinc ricinoleate at greater than 200 Pa-s (composition D) and 60 Pa-s (preparation C).

Such high viscosities are hardly processible in practice but the advantage of V1 which contributes to lower viscosities is apparent.

Viscosities of less than 10 000 mPa-s or better less than 3000 mPa-s are advantageous for good pumpability.

The advantage of V1 over EDA-PO is also apparent from preparations such as F and G. A liquid product having a viscosity of 2100 mPa-s is markedly easier to process than a material of 8800 mPa-s.

Using V1 as a mixture component made it possible to have zinc acetate dihydrate in a mixture in proportions of 45% and a viscosity of 7000 mPa-s as is apparent in composition J; i.e. lower viscosity than a mixture such as G comprising 30% zinc acetate dihydrate.

Despite a relatively low viscosity of less than 1000 mPa-s preparation L showed precipitation after several days. By contrast, preparation K containing V1 instead of EPA-PO remained a stable clear solution for weeks.

It is clearly apparent that the inventive preparations have advantages over the compositions known hitherto.

Additional Trimerization Catalysts that May be Part of a Composition:

Component M: Potassium acetate-based trimerization catalyst: Kosmos® 33 MEG from Evonik Operations GmbH.

Component N: Potassium octoate-based trimerization catalyst: Kosmos® 75 from Evonik Operations GmbH.

Component O: Potassium neodecanoate-based trimerization catalyst: Kosmos® K 65 LO from Evonik Operations GmbH

Component P: Potassium propionate-based trimerization catalyst: KPROP 14 from Schill&Seilacher

### Production of PU and PIR Foams:

Foaming was carried out by manual mixing. For this purpose, the compounds according to the invention, polyols, flame retardants, catalysts according to the invention or not according to the invention, water, siloxane surfactant and blowing agent were weighed into a beaker and mixed by means of a disc stirrer (diameter 6 cm) at 1000 rpm for 30 s. The beaker was reweighed to determine the amount of blowing agent that had evaporated during the mixing operation and this was replenished. Subsequently, the isocyanate (MDI) was added, and the reaction mixture was stirred with the stirrer described at 3000 rpm for 5 s.

The reaction mixtures were introduced into appropriate beakers having a diameter at the upper edge of 20 cm in order to obtain free-rise foams. The amount of the reaction mixture was chosen such that the tip of the foam dome at the end was 10 to 15 cm above the upper edge of the beaker.

During the foaming, the gel time was determined, in order to assess the influence of the catalysts on the speed of foaming.

After 2 minutes, the foam domes were cut off at the upper edge of the beaker, such that a round foam surface was obtained. The indentation hardnesses of the foams were determined at this surface.

### Method of Determining Indentation Hardness:

For this purpose, the force for indenting a die of diameter 4 cm into the foam was measured. The indentation forces were measured at indentation depth 5 mm. Measurement was carried out at 90 second intervals, i.e. after 3, 5½, 8 and 10½ minutes, wherein the die was indented at 4 different points on the cut surface in a circular arrangement.

### Method of Determining Compression Hardness:

The compressive strengths of the foams are measured on cubic test specimens having an edge length of 5 cm in accordance with DIN EN ISO 844:2014-11 up to a compression of 10% (the maximum compressive stress occurring in this measuring range is reported).

Table 2 summarizes the employed foam formulations (form. 1 to form. 9). The respective weight fractions of the constituents are reported.

These formulations were then admixed with further catalysts, then accordingly corresponding to inventive or noninventive compositions. These further catalysts and the results therefor are then summarized in table 3.

TABLE 2
Foam formulations (PIR and PUR)

| Formulation | Form. 1 | Form. 2 | Form. 3 | Form. 4 | Form. 5 |
|---|---|---|---|---|---|
| Daltolac ® R 471 | | | | | |
| PS 2352 | 100 | 100 | 100 | 100 | 100 |
| POLYCAT ® 5 | 0.5 | | | | |
| POLYCAT ® 9 | | 0.55 | | | |
| POLYCAT ® 206 | | | 0.9 | | |
| POLYCAT ® 77 | | | | 0.65 | |
| TEGOAMIN ® BDE | | | | | 0.8 |
| Further cats. | see | see | see | see | see |
| | table 3 | table 3 | table 3 | table 3 | table 3 |
| TEGOSTAB ® B 8460 | 2 | 2 | 2 | 2 | 2 |
| TCPP | 15 | 15 | 15 | 15 | 15 |
| Water | 0.5 | 0.5 | 0.5 | 0.5 | 0.5 |
| n-Pentane | 14 | 14 | 14 | 14 | 14 |
| Cyclopentane | | | | | |
| MDI (44V20) | about 190 | about 190 | about 190 | about 190 | about 190 |
| Index | 255 | 255 | 255 | 255 | 255 |

Formulations

| Formulation | Form. 6 | Form. 7 | Form. 8 | Form. 9 | Form. 10 | Form. 11 |
|---|---|---|---|---|---|---|
| Daltolac ® R 471 | | | | | 100 | 100 |
| PS 2352 | 100 | 100 | 100 | 100 | | |
| POLYCAT ® 5 | | | | | 2.1 | |
| POLYCAT ® DP | | | | | | 3.5 |
| DABCO ® T | 0.5 | | | | | |
| POLYCAT ® NE 300 | | 1.1 | | | | |
| Amine no. 1 | | | 1 | | | |
| Amine no. 2 | | | | 1.1 | | |
| Further cats. | see | see | see | see | see | see |
| | table 3 | table 3 | table 3 | table 3 | table 3 | table 3 |
| TEGOSTAB ® B 8460 | 2 | 2 | 2 | 2 | 1.4 | 1.4 |
| TCPP | 15 | 15 | 15 | 15 | | |
| Water | 0.5 | 0.5 | 0.5 | 0.5 | 2.5 | 2.5 |
| n-Pentane | 14 | 14 | 14 | 14 | | |
| Cyclopentane | | | | | 13 | 13 |
| MDI (44V20) | about 190 | about 190 | about 190 | about 190 | about 190 | about 190 |
| Index | 255 | 255 | 255 | 255 | 130 | 130 |

### Foaming Results:

### Table 3:

Summary of foaming experiments with various catalysts and foam formulations.

The table reports the employed components (comp. A-P, inventive or noninventive according to the composition), the dosages thereof (parts by weight) (referred to as dos.), the employed formulation from table 2, the gel time (GT) in seconds, and the indentation hardnesses in newtons after the time specified in minutes (after mixing with MDI).

The catalyst compositions ZZ, which contain either no V1 or no zinc, are noninventive.

The components of trimerization catalysts, amines and ZZ may also be premixed. For clarity, in the examples here the components such as for example tert. amines, zinc salts, nitrogen-containing compound V1) and trimerization catalysts are reported via the formulations in table 2 and the additional components are reported separately in table 3.

TABLE 3

| | | | | | | | Force in N after time of | | | |
|---|---|---|---|---|---|---|---|---|---|---|
| | | | | | Form. | | 3 | | 8 | 10′30″ |
| Ex. | Cmp. | Dos. | Cmp. | Dos. | No. | GT/sec. | min | 5′30′″ | min | min |
| 1 | K | 1 | M | 1.4 | 1 | 61 | 121 | 260 | 367 | 424 |
| Comp. | | | M | 1.1 | 1 | 64 | 85 | 194 | 284 | 375 |
| ex. 1 | | | | | | | | | | |
| Comp. | L | 1 | M | 2 | 1 | 59 | 112 | 260 | 365 | 430 |
| ex. 2 | | | | | | | | | | |
| 2 | K | 2 | M | 2 | 1 | 59 | 219 | 328 | 413 | 479 |
| 3 | E | 1 | M | 1.1 | 1 | 61 | 96 | 258 | 329 | 394 |
| 4 | A | 1 | M | 0.9 | 1 | 61 | 72 | 192 | 286 | 373 |
| 5 | C | 1 | M | 1.1 | 1 | 60 | 75 | 226 | 311 | 385 |
| 6 | J | 0.5 | M | 1.3 | 1 | 65 | 87 | 258 | 334 | 421 |
| 7 | J | 0.3 | M | 1.2 | 1 | 62 | 102 | 248 | 335 | 407 |
| 8 | J | 0.25 | P | 1.0 | 1 | 63 | 86 | 234 | 415 | 454 |
| 9 | J | 0.425 | O | 1.7 | 1 | 61 | 109 | 256 | 346 | 396 |
| Comp. | | | M | 1.1 | 2 | 60 | 75 | 183 | 256 | 355 |
| ex. 3 | | | | | | | | | | |
| 10 | K | 1 | M | 1.4 | 2 | 61 | 114 | 236 | 354 | 412 |
| 11 | J | 0.5 | M | 1.2 | 2 | 60 | 118 | 232 | 356 | 424 |
| Comp. | | | M | 1.1 | 3 | 61 | 83 | 190 | 267 | 371 |
| ex. 4 | | | | | | | | | | |
| 12 | K | 1 | M | 1.4 | 3 | 61 | 123 | 253 | 347 | 413 |
| 13 | J | 0.5 | M | 1.2 | 3 | 60 | 119 | 261 | 364 | 418 |
| Comp. | | | M | 1.1 | 4 | 59 | 93 | 189 | 284 | 362 |
| ex. 5 | | | | | | | | | | |
| 14 | K | 1 | M | 1.4 | 4 | 61 | 132 | 235 | 376 | 435 |
| 15 | J | 0.5 | M | 1.2 | 4 | 59 | 127 | 263 | 356 | 419 |
| Comp. | | | M | 1.1 | 5 | 60 | 87 | 198 | 273 | 367 |
| ex. 6 | | | | | | | | | | |
| 16 | K | 1 | M | 1.4 | 5 | 60 | 117 | 259 | 361 | 421 |
| 17 | J | 0.5 | M | 1.2 | 5 | 60 | 116 | 264 | 365 | 419 |
| Comp. | | | M | 1.1 | 6 | 61 | 85 | 187 | 279 | 363 |
| ex. 7 | | | | | | | | | | |
| 18 | K | 1 | M | 1.4 | 6 | 61 | 120 | 269 | 371 | 420 |
| 19 | J | 0.5 | M | 1.2 | 6 | 59 | 124 | 270 | 359 | 409 |
| Comp. | | | M | 1.1 | 7 | 60 | 87 | 191 | 275 | 369 |
| ex. 8 | | | | | | | | | | |
| 20 | K | 1 | M | 1.4 | 7 | 60 | 130 | 258 | 373 | 424 |
| 21 | J | 0.5 | M | 1.2 | 7 | 61 | 126 | 266 | 365 | 418 |
| Comp. | | | M | 1.1 | 8 | 60 | 93 | 199 | 283 | 371 |
| ex. 9 | | | | | | | | | | |
| 22 | K | 1 | M | 1.4 | 8 | 59 | 125 | 265 | 371 | 422 |
| 23 | J | 0.5 | M | 1.2 | 8 | 60 | 131 | 259 | 378 | 433 |
| Comp. | | | M | 1.1 | 9 | 60 | 88 | 194 | 277 | 368 |
| ex. 10 | | | | | | | | | | |
| 24 | K | 1 | M | 1.4 | 9 | 61 | 134 | 267 | 369 | 429 |
| 25 | J | 0.5 | M | 1.2 | 9 | 61 | 122 | 271 | 372 | 428 |
| Comp. | | | | | 10 | 56 | 72 | 151 | 234 | 309 |
| ex. 11 | | | | | | | | | | |
| 26 | K | 1 | | | 10 | 56 | 172 | 281 | 348 | 411 |
| Comp. | | | | | 11 | 52 | 107 | 183 | 255 | 366 |
| ex. 12 | | | | | | | | | | |
| 27 | K | 1 | | | 11 | 44 | 244 | 362 | 420 | 445 |

Example 1 achieved markedly higher indentation hardnesses than comp. ex. 1 where no inventive zinc-containing composition was used.

The advantage of the inventive composition is also apparent from comp. ex. 2. Combination with L (noninventive) requires the use of 2 parts of M to achieve the same gel time and through-curing as example 1. Here, a gel time of 60 seconds and comparable through-curing/indentation hardnesses are achievable with 1.4 parts of M.

K is thus a more efficient catalyst than L.

Example 7 employed only 0.3 parts of J and 1.2 parts of Kosmos 33 to achieve a gel time of 60 seconds and the improved indentation hardnesses.

This demonstrates the advantage that the zinc-phenol combinations make it possible to provide more efficient catalysts for through-curing.

P and O also achieved the desired effects on foam hardness with small dosages of J as is apparent from examples 8 and 9.

The further experiments in different formulations show the same effect.

It is apparent therefrom that the invention makes it possible to achieve improved curing of the foam in a very wide variety of formulations. It is even possible here in some cases to prolong the gel times, or to further improve the positive effects on through-curing with equal gel times.

This is an enormous advantage since, by virtue of the minor influence on gel time, the processability of the reaction mixture is maintained, for example with regard to the flowability of the foaming mixture, and the curing of the foam is simultaneously accelerated.

It is clearly apparent from the experiments that the inventive zinc-containing preparations/inventive compositions lead to improved curing of the foam. The above-described very good results for the indentation hardnesses of the inventive foams correspond to those for compressive strength.

1. A composition for producing polyurethane or polyisocyanurate foam, the composition comprising:
a) a polyisocyanate component,
b) a polyol component,
c) at least one zinc(II) carboxylate with the proviso that the zinc(II) carboxylate present is employed in stoichiometric form,
d) at least one tertiary amine of formula (X) 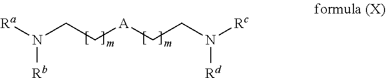
where
m is independently at each occurrence 1 or 2,
A is O, S or N—R^{e},
R^{a}, R^{b}, R^{c}, R^{d} and R^{e} are each independently of one another identical or different linear, branched or cyclic alkyl radicals having 1 to 20 carbon atoms,
e) optionally at least one foam stabilizer,
f) optionally at least one blowing agent,
wherein the composition additionally contains
g) at least one nitrogen-containing compound V which comprises at least two N atoms and which is a modified phenol.

2. The composition according to claim 1, wherein the at least one zinc(II) carboxylate is selected from the group consisting of zinc(II) acetate, zinc(II) propionate, zinc(II) pivalate, zinc(II) 2-ethylhexanoate (zinc(II) octoate), zinc(II) isononanoate (zinc(II) 3,5,5-trimethylhexanoate), zinc(II) neodecanoate, zinc(II) ricinoleate, zinc(II) palmitate, zinc(II) stearate, zinc(II) oleate, zinc(II) laurate, zinc(II) naphthenate, zinc(II) benzoate, zinc(II) lactate, zinc(II) glycinate, zinc(II) hippurate, zinc(II) citrate and zinc(II) soaps.

3. The composition according to claim 1, wherein the at least one nitrogen-containing compound V is selected from the group consisting of 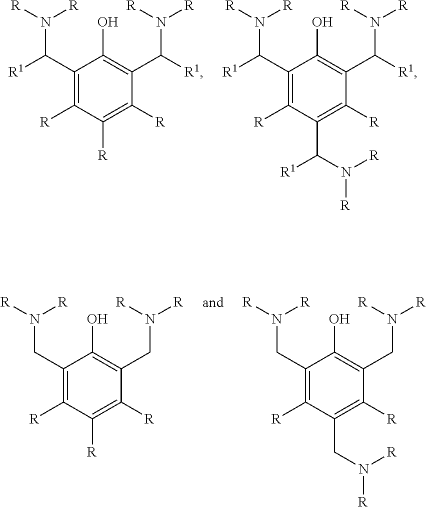
wherein
R=independently at each occurrence H or linear, branched or cyclic hydrocarbon radical having 1 to 20 carbon atoms which is optionally saturated, unsaturated or aromatic, optionally containing heteroatoms such as O or N,
R¹=independently at each occurrence H or linear, branched or cyclic hydrocarbon radical having 1 to 20 carbon atoms which is optionally saturated, unsaturated or aromatic.

4. The composition according to claim 1, wherein the at least one nitrogen-containing compound V is produced by reaction of phenol with aldehyde,
wherein the starting material employed is optionally selected from modified phenols bearing at least one substituent on the aromatic core,
wherein the starting material employed is optionally selected from modified phenols.

5. The composition according to claim 1, wherein the at least one nitrogen-containing compound V is selected from the group consisting of 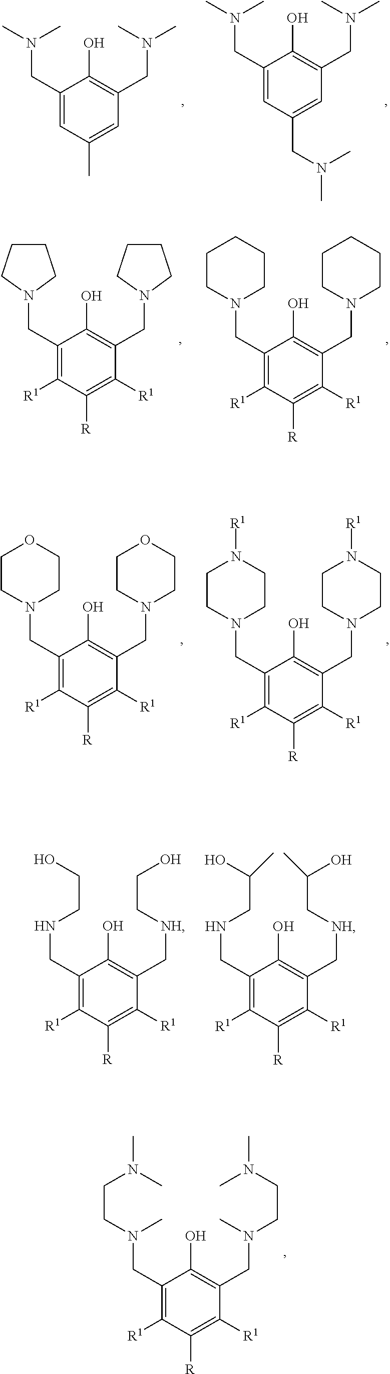 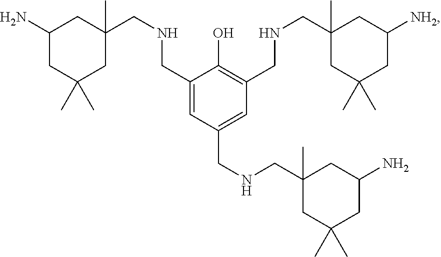 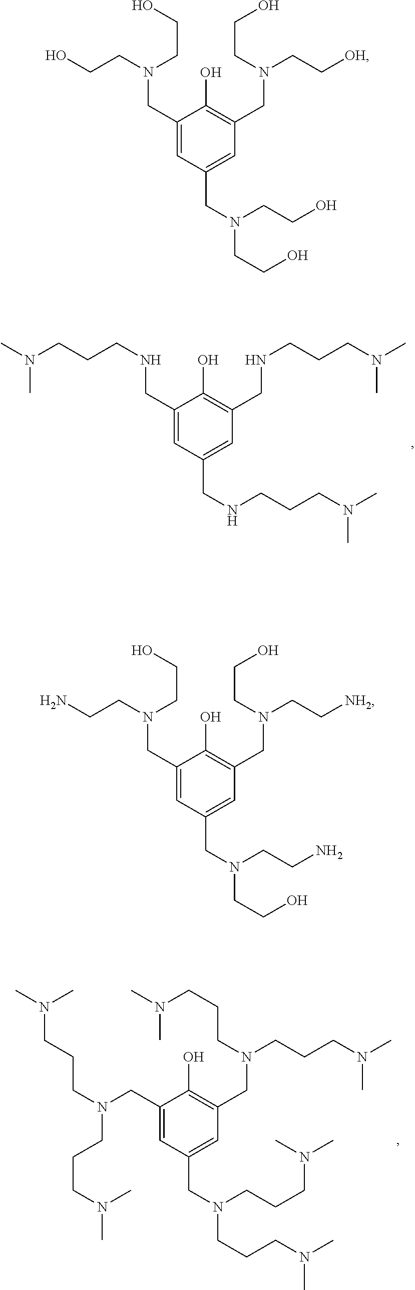 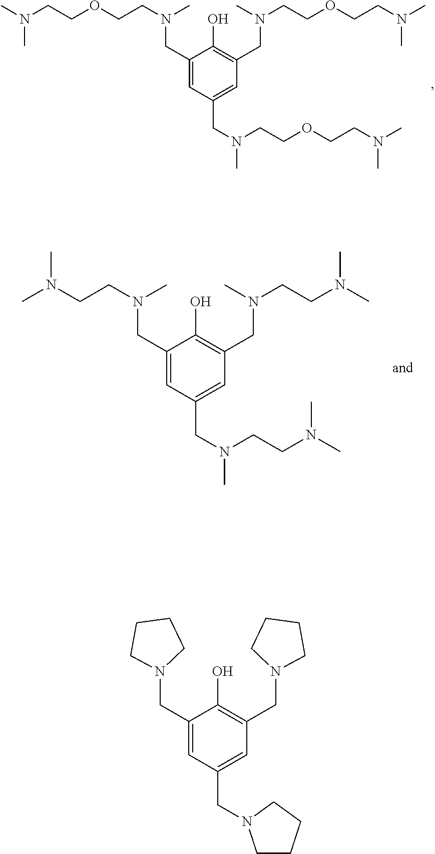
wherein
R=independently at each occurrence H or linear, branched or cyclic hydrocarbon radical having 1 to 20 carbon atoms which is optionally saturated, unsaturated or aromatic, optionally containing heteroatoms such as O or N,
R¹=independently at each occurrence H or linear, branched or cyclic hydrocarbon radical having 1 to 20 carbon atoms which is optionally saturated, unsaturated or aromatic.

6. The composition according to claim 1, wherein the composition contains altogether present zinc(II) carboxylate and altogether present nitrogen-containing compound V in a quantity ratio of 1:0.5 to 1:5 parts by weight relative to one another.

7. The composition according to claim 1, wherein the at least one tertiary amine of formula (X) is selected from the group consisting of pentamethyldiethylenetriamine, bis(2-dimethylaminoethyl) ether, tris(dimethylaminopropyl)amine, N-[2-[2-(dimethylamino)ethoxy]ethyl]-N-methyl-1,3-propanediamine, diisopropyltrimethyldiethylenetriamine, bis(dimethylaminopropyl)methylamine, trimethylaminoethylethanolamine, bis(2-isopropylmethylaminoethyl) ether, bis(2-isobutylmethylaminoethyl) ether, N,N′-diisopropyl-N,N′-dimethyl-bis(aminoethyl) ether, N,N,N′-triisopropyl-N′-methyl-bis(aminoethyl) ether and N,N′-diisobutyl-N,N′-dimethyl-bis(aminoethyl) ether.

8. The composition according to claim 1, wherein the composition additionally contains at least one additional trimerization catalyst.

9. The composition according to claim 1, wherein the altogether present polyol component consists to an extent of 70% to 100% by weight of aromatic polyester polyols having OH numbers of 150 to 400 mg KOH/g.

10. The composition according to claim 1, wherein the composition comprises at least one physical blowing agent.

11. A Zinc(II) carboxylate-containing preparation suitable for catalysis in the production of PU or PIR foam, the preparation comprising:
i) at least one zinc(II) carboxylate, in a total amount of 2% to 50% by weight, with the proviso that the zinc(II) carboxylate present is employed in stoichiometric form,
ii) optionally at least one carrier medium in a total amount of 0% to 95% by weight,
iii) at least one nitrogen-containing compound V which comprises at least two N atoms and which is a modified phenol, in a total amount of 1% to 90% by weight,
in each case based on the total preparation.

12. A process for producing polyurethane or polyisocyanurate foam, the process comprising:
reacting a polyol component with a polyisocyanate component, using a composition according to claim 1.

13. A polyurethane or polyisocyanurate foam obtained by the process according to claim 12.

14. A process, comprising:
installing a polyurethane or polyisocyanurate foam according to claim 13 for thermal insulation purposes.

15. The composition according to claim 2, wherein the at least one zinc(II) carboxylate is selected from the group consisting of zinc(II) acetate and zinc(II) ricinoleate.

16. The composition according to claim 4, wherein the at least one nitrogen-containing compound V is produced by reaction of phenol with at least one aldehyde selected from the group consisting of formaldehyde, acetaldehyde, propionaldehyde, butyraldehyde, benzaldehyde, cinnamaldehyde, trimethylhexanal, pelargonaldehyde, acrolein, furfural and amine in the context of a Mannich reaction, and aldehydes having 1 to 20 carbon atoms.

17. The composition according to claim 4, wherein the starting material employed is at least one selected from the group consisting of cresol, xylenols, propylphenols, styrylphenols, alkylphenols, cardanol, and modified phenols bearing at least one substituent on the aromatic core.

18. The composition according to claim 4, wherein the at least one nitrogen-containing compound V is produced by reaction of phenol with formaldehyde and at least one primary or secondary amine selected from the group consisting of dimethylamine, diethylamine, dipropylamine, diisopropylamine, dibutylamine, ethanolamine, diethanolamine, trimethylethanediamine, isophoronediamine, dimethylpropylamine, diethylenetriamine, triethylenetetramine, methylhydroxyethylamine, dimethylaminopropylamine, pyrrolidine, pyrrole, morpholine and/or piperazine, N,N-dimethyl-2-(2-methylaminoethoxy)ethan-1-amine, N-[2-[2-(dimethylamino)ethoxy]ethyl]-N-methyl-1,3-propanediamine, diisopropyldimethyldiethylenetriamine, bis(dimethylaminopropyl)amine, trimethylaminoethylethanolamine, N,N′-diisopropyl-N-methyl-bis(aminoethyl) ether, and N,N′-diisobutyl-N-methyl-bis(aminoethyl) ether.

19. The composition according to claim 8, wherein the at least one trimerization catalyst is at least one carboxylate selected from the group consisting of ammonium carboxylate, potassium carboxylate, and other alkali and alkali earth metal carboxylates.

20. The composition according to claim 8, wherein the at least one trimerization catalyst is at least one carboxylate selected from the group consisting of potassium acetate, potassium formate, potassium propionate, potassium butanoate, potassium pentanoate, potassium hexanoate, potassium heptanoate, potassium 2-ethylhexanoate, potassium pivalate, potassium octoate, potassium butyrate, potassium isobutyrate, potassium nonanoate, potassium decanoate, potassium ricinoleate, potassium stearate, potassium neodecanoate and carboxylates of tetramethylammonium, tetraethylammonium, triethylmethylammonium, tetrapropylammonium, tetrabutylammonium, dimethyldiallylammonium, trimethyl-(2-hydroxypropyl)ammonium, triethyl-(2-hydroxypropyl)ammonium, tripropyl-(2-hydroxypropyl)ammonium, tributyl-(2-hydroxypropyl)ammonium, trimethyl-(2-hydroxyethyl)ammonium, triethyl-(2-hydroxyethyl)ammonium, tripropyl-(2-hydroxyethyl)ammonium, tributyl-(2-hydroxyethyl)ammonium, dimethylbenzyl-(2-hydroxyethyl)ammonium and dimethylbenzyl-(2-hydroxypropyl)ammonium.
